(19) United States
(12) Patent Application Publication
(10) Pub. No.: US 2023/0240551 A1
(43) Pub. Date: Aug. 3, 2023

(54) BIOMETRIC INFORMATION ACQUISITION METHOD, PROGRAM, AND BIOMETRIC INFORMATION ACQUISITION METHOD

(71) Applicant: Panasonic Intellectual Property Management Co. Ltd, Osaka (JP)

(72) Inventor: Nobuyuki OTSUKA

(21) Appl. No.: 17/909,945

(22) PCT Filed: Jan. 12, 2021
(86) PCT No.: PCT/JP2021/000724

§ 371 Date: Sep. 7, 2022

(30) Foreign Application Priority Data
Mar. 27, 2020 (JP) 2020-059105

(51) Int. Cl. A61B 5/0537; A61N 1/36

(52) U.S. Cl. CPC A61B 5/0537; A61N 1/36003; A61N 1/36034

(57) ABSTRACT

A biometric information acquisition method according to the present disclosure includes an application step and an acquisition step. The application step includes applying an electrical signal to an organism’s muscle. The acquisition step includes acquiring information about the organism’s muscle as biometric information based on a change rate with respect to an impedance of the electrical signal applied in the application step.

### TECHNICAL FIELD

The present disclosure generally relates to a biometric information acquisition method, a program, and a biometric information acquisition system. More particularly, the present disclosure relates to a biometric information acquisition method for acquiring biometric information about an organism by applying an electrical signal to the organism, a program designed to perform the biometric information acquisition method, and a biometric information acquisition system for acquiring biometric information about an organism by applying an electrical signal to the organism.

### BACKGROUND ART

Patent Literature 1 discloses a biometric information acquisition apparatus. The biometric information acquisition apparatus includes four electrodes for measuring a bioelectrical impedance of a subject standing on the body of the apparatus. These electrodes are connected to an electronic circuit including an integrated circuit for calculating the subject’s body composition, for example. The electronic circuit calculates the subject’s bioelectrical impedance by using the electrodes and eventually calculates the subject’s body composition based on the bioelectrical impedance thus calculated, the subject’s weight calculated using a load cell, the subject’s gender that has been preset, and other data. As the body composition, a muscle level indicating the muscle mass, for example, is calculated.

### CITATION LIST

### Patent Literature

Patent Literature 1: JP 2012-200276 A

### SUMMARY OF INVENTION

It is therefore an object of the present disclosure to provide a biometric information acquisition method, a program, and a biometric information acquisition system, all of which make it easier to recognize a change in an organism’s muscles.

A biometric information acquisition method according to an aspect of the present disclosure includes an application step and an acquisition step. The application step includes applying an electrical signal to an organism’s muscle. The acquisition step includes acquiring information about the organism’s muscle as biometric information based on a change rate with respect to an impedance of the electrical signal applied in the application step.

A program according to another aspect of the present disclosure is designed to cause one or more processors to perform the biometric information acquisition method described above.

A biometric information acquisition system according to still another aspect of the present disclosure includes an application unit and an acquisition unit. The application unit applies an electrical signal to an organism’s muscle. The acquisition unit acquires information about the organism’s muscle as biometric information based on a change rate with respect to an impedance of the electrical signal applied by the application unit.

### BRIEF DESCRIPTION OF DRAWINGS

FIG. 1 is a block diagram schematically illustrating a biometric information acquisition system as an agent that performs a biometric information acquisition method according to an exemplary embodiment of the present disclosure;

FIG. 2 illustrates a phase angle of an impedance of an electrical signal;

FIG. 3 is a schematic representation illustrating a state where electrodes are attached to an organism;

FIG. 4 shows an exemplary correlation between a change with time in the phase angle of the impedance of an electrical signal and the frequency of the electrical signal in the biometric information acquisition system;

FIG. 5 shows an exemplary correlation between the phase angle and phase angle change rate of the impedance of an electrical signal and the frequency in the biometric information acquisition system;

FIG. 6 shows an exemplary change with time in the phase angle change rate of the impedance of an electrical signal in the biometric information acquisition system; and

FIG. 7 is a flowchart showing an exemplary procedure of operation of the biometric information acquisition system.

### DESCRIPTION OF EMBODIMENTS

### Overview

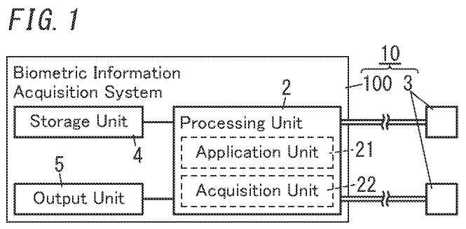

Next, a biometric information acquisition method according to this embodiment and a biometric information acquisition system 100 (see FIG. 1), which is an agent that performs the biometric information acquisition method, will be described with reference to the accompanying drawings. Note that the embodiment to be described below is only an exemplary one of various embodiments of the present disclosure and should not be construed as limiting. Rather, the exemplary embodiment may be readily modified in various manners depending on a design choice or any other factor without departing from the scope of the present disclosure. The drawings to be referred to in the following description of embodiments are all schematic representations. That is to say, the ratio of the dimensions (including thicknesses) of respective constituent elements illustrated on the drawings does not always reflect their actual dimensional ratio.

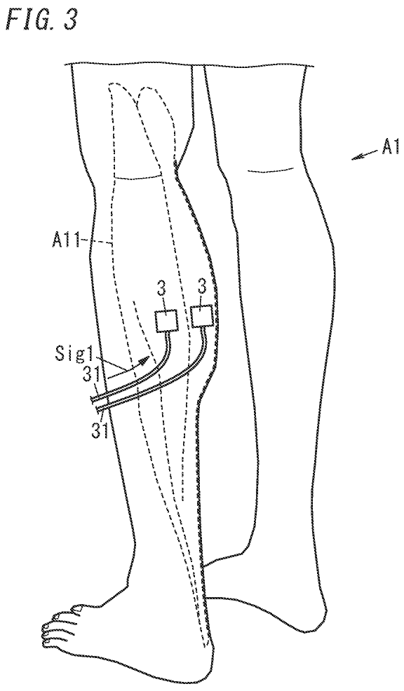

A biometric information acquisition method (biometric information acquisition system 100) according to this embodiment is a method (or system) for acquiring information about an organism A1 (in particular, biometric information about the organism’s A1 muscle A11) by applying an electrical signal to the organism’s A1 (see FIG. 3) muscle A11. In this embodiment, the organism A1 is a human being. Naturally, the organism A1 may also a non-human creature such as an animal. Also, in this embodiment, the muscle A11 is not the entire muscles of the human being but a particular muscle that forms part of the human being’s muscles. In this embodiment, the muscle A11 may be, for example, a gastrocnemius muscle.

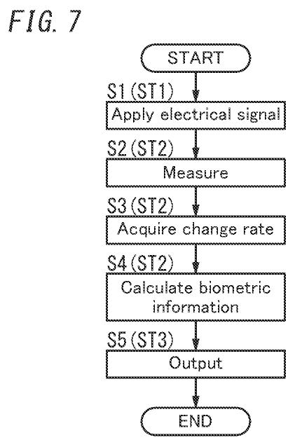

As shown in FIG. 1, the biometric information acquisition system 100 includes an application unit 21 and an acquisition unit 22. The application unit 21 is an agent that performs an application step ST1 (see FIG. 7) of the biometric information acquisition method according to this embodiment. The acquisition unit 22 is an agent that performs an acquisition step ST2 (see FIG. 7) of the biometric information acquisition method according to this embodiment.

The application unit 21 performs, as the application step ST1, processing of applying an electrical signal Sig1 to the organism’s A1 muscle A11. In this embodiment, the application unit 21 applies (i.e., the application step ST1 includes applying) the electrical signal Sig1 to the organism’s A1 muscle A11 via a pair of electrodes 3 as shown in FIG. 3. Specifically, the application unit 21 applies the electrical signal Sig1 to the organism’s A1 muscle A11 by applying an AC voltage between the pair of electrodes 3.

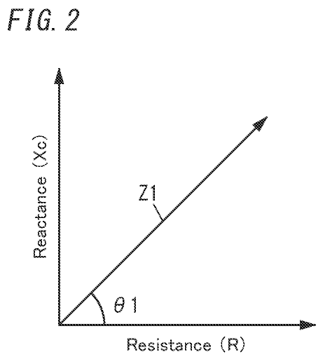

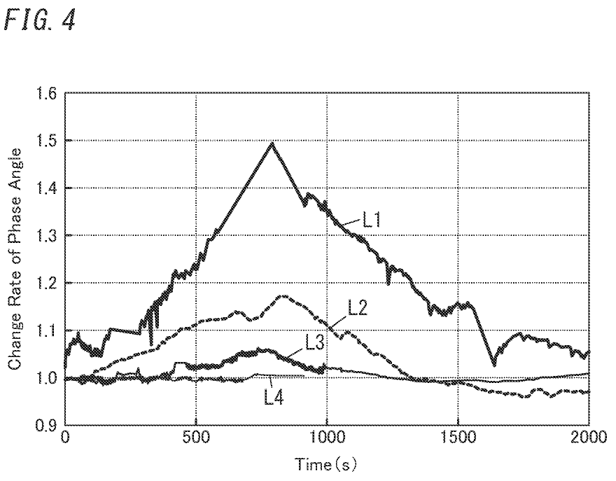

The acquisition unit 22 performs, as the acquisition step ST2, processing of acquiring information about the organism’s A1 muscle A11 as biometric information based on a change rate D1 (see FIG. 4) with respect to an impedance Z1 (see FIG. 2) of the electrical signal Sig1 applied by the application unit 21 (in the application step ST1). In this embodiment, the electrical signal Sig1 is applied to the organism’s A1 muscle A11 via the pair of electrodes 3 as described above. Thus, the acquisition unit 22 acquires the change rate D1 with respect to the impedance Z1 of the electrical signal Sig1 by measuring the electrical signal Sig1 applied to the organism’s A1 muscle A11 via the pair of electrodes 3. As used herein, the “electrical signal Sig1” includes a voltage signal applied between the pair of electrodes 3 and a current signal flowing through the organism A1 upon the application of the voltage. Also, the “impedance Z1 of the electrical signal Sig1” as used herein refers to the organism’s A1 impedance (e.g., bioelectrical impedance) obtained based on the voltage signal and current signal of the electrical signal Sig1.

In this paragraph, the mechanism of a muscle mass increase in an organism’s A1 muscle A11 will be described briefly. In general, as a load is applied to an organism’s A1 muscle A11 as the organism A1 exercises, damage is done to its muscle fibers to varying degrees according to the magnitude and duration of the load applied. Then, muscle swelling occurs in the damaged region. When muscle swelling occurs, the amount of fluid (specifically, the volume of the interstitial fluid, in particular, out of the extracellular fluid) increases locally in the damaged region. Thereafter, the damaged muscle fibers are going to recover their original condition with the passage of time. In the meantime, the muscle fibers may be enlarged compared to its original condition, i.e., so-called “supercompensation” may occur. In this manner, the muscle mass of the organism’s A1 muscle A11 may vary as the organism A1 exercises.

The present inventors discovered via experiments that the degree of muscle swelling (degree of swelling) of the muscle A11 is correlated to the product of the change rate D1 of the impedance Z1 of the electrical signal Sig1 (in particular, its phase angle θ1 (see FIG. 2)) and the duration of the exercise done by the organism A1. It is also known in the art that there is positive correlation between the degree of swelling of the muscle A11 and an increase in the muscle mass of the muscle A11. Thus, the acquisition unit 22 may acquire (i.e., the acquisition step ST2 may include acquiring) information about an organism’s A1 muscle A11 (e.g., information about the muscle mass of the muscle A11 in this example) as biometric information by acquiring a change rate D1 with respect to the impedance Z1 of the electrical signal Sig1. In the following description of an exemplary embodiment, the change rate D1 is supposed to refer to the change rate D1 of the phase angle θ1 of the impedance Z1 of the electrical signal Sig1 (i.e., a so-called “phase shift rate”) unless otherwise stated.

As described above, in this embodiment, the electrical signal Sig1 is applied to the organism’s A1 muscle A11 and a change rate D1 with respect to the impedance Z1 of the electrical signal Sig1, which is known to have positive correlation with the muscle mass of the muscle A11, is acquired. Therefore, this embodiment enables acquiring, based on this change rate D1, a parameter correlated to the muscle mass of the organism’s A1 muscle A11, thus eventually achieving the advantage of making it easier to recognize a change in the organism’s A1 muscle A11.

### Details

Next, a biometric information acquisition system 100 according to this embodiment will be described in detail with reference to FIG. 1. In this embodiment, the biometric information acquisition system 100 is supposed to form, along with a pair of electrodes 3, a measuring apparatus 10. In other words, in this embodiment, respective constituent elements of the biometric information acquisition system 100 are supposed to be housed in the housing of the measuring apparatus 10 except the pair of electrodes 3.

The biometric information acquisition system 100 includes a processing unit 2, a storage unit 4, and an output unit 5. In this embodiment, the storage unit 4 is counted among the constituent elements of the biometric information acquisition system 100. However, this is only an example and should not be construed as limiting. Alternatively, the storage unit 4 may be counted out of its constituent elements.

Each of the pair of electrodes 3 is provided at one end of an associated one of two cables 31 extended from the housing of the measuring apparatus 10. One surface of each of the pair of electrodes 3 is an adhesive pad to allow the electrode 3 to be adhered to the skin surface of the organism A1. Thus, adhering the pair of electrodes 3 to the organism’s A1 skin surface that faces the muscle A11, from which the biometric information needs to be acquired, as shown in FIG. 3 makes the electrical signal Sig1 ready to be applied to the muscle A11 via the pair of electrodes 3. In the example shown in FIG. 3, the pair of electrodes 3 are adhered to the skin surface that faces the organism’s A1 gastrocnemius muscle (muscle A11) (i.e., to the skin surface of his or her calf). Note that the pair of electrodes 3 may be either adhered laterally (horizontally) to a plurality of muscles as shown in FIG. 3 or adhered longitudinally (vertically) to a single muscle. Adhering the pair of electrodes 3 longitudinally achieves the advantage of enabling acquiring biometric information locally from only one muscle.

Applying the electrical signal Sig1 to the muscle A11 using the pair of electrodes 3 as described above allows the electrodes 3 to be placed in a narrower region than using three or more electrodes 3. Thus, using the pair of electrodes 3 allows the electrodes 3 to be placed onto the muscle A11 to increase the change rate D1 of a value concerning the impedance Z1 (e.g., the phase angle θ1 in this example) compared to using three or more electrodes 3. In addition, using the pair of electrodes 3 also achieves the advantages of increasing the degree of adhesiveness of the electrodes 3 to the muscle A11 and simplifying the measurement, compared to using three or more electrodes 3.

The processing unit 2 is a computer system including one or more processors and a memory as principal hardware components thereof. This processing system 2 may perform various functions by making the one or more processors execute a program stored in the memory. The program may be stored in advance in the memory of the processing unit 2. Alternatively, the program may also be downloaded through a telecommunications line or be distributed after having been recorded in some non-transitory storage medium such as an optical disc or a hard disk drive, both of which are readable for the computer system.

The processing unit 2 includes the application unit 21 and the acquisition unit 22.

The application unit 21 performs, as the application step ST1, processing of applying the electrical signal Sig1 to the organism’s A1 muscle A11. In this embodiment, the application unit 21 applies an AC voltage as the electrical signal Sig1 to the pair of electrodes 3, thereby applying the AC voltage to the organism’s A1 muscle A11. More specifically, in this embodiment, the application unit 21 applies (i.e., the application step ST1 includes applying), as the electrical signal Sig1, either a sinusoidal voltage or a pulse-modulated voltage to the organism’s A1 muscle A11. As used herein, the “pulse-modulated voltage” refers to a pulse voltage, of which the fundamental harmonic may have the frequency of a sinusoidal wave, for example.

Applying a pulse-modulated voltage to the organism’s A1 muscle A11 as described above causes the electrolyte (such as potassium or calcium) included in the muscle’s A11 cells to move. This phenomenon is caused because the electrolyte in the cells, which is cations, is attracted to a negative electrode. Thus, applying a pulse-modulated voltage to the organism’s A1 muscle A11 achieves the advantage of making it easier to measure a peak of the change rate D1 of a value concerning the impedance Z1 (e.g., the phase angle θ1) than applying a sinusoidal AC voltage. This is because causing the electrolyte to move in advance and be attracted toward the negative electrode reduces the chances of the electrolyte to move during the measurement. In other words, this reduces the chances of the movement of the electrolyte obstructing the measurement.

Also, in this embodiment, the electrical signal Sig1 has a single frequency. In other words, the frequency of the electrical signal Sig1 does not change while the impedance Z1 is being measured. Setting the frequency of the electrical signal Sig1 at a single value in this manner achieves the advantages of making it easier to simplify the measuring range, shorten the measuring time, simplify the analysis of the measured data, and simplify the configuration of the measuring apparatus 10, compared to allowing the frequency to vary.

Furthermore, in this embodiment, the electrical signal Sig1 is a voltage on the order of mV. That is to say, in this embodiment, an electric current on the order of mA is caused to flow through the organism’s A1 muscle A11 by applying an AC voltage on the order of mV to the pair of electrodes 3. Thus, this embodiment achieves the advantage of reducing the chances of the biometric information being affected by brain waves with a voltage on the order of µV (i.e., an electric current on the order of µA) while the biometric information is being acquired.

The acquisition unit 22 performs, as the acquisition step ST2, processing of acquiring information about the organism’s A1 muscle A11 as biometric information based on a change rate D1 with respect to the impedance Z1 of the electrical signal Sig1 applied by the application unit 21. In this embodiment, the change rate D1 with respect to the impedance Z1 is the change rate D1 of the phase angle θ1 of the impedance Z1 as described above.

Specifically, the acquisition unit 22 obtains a change with time in the impedance Z1 (in particular, a change with time in the phase angle θ1 of the impedance Z1) by measuring the impedance Z1 of the electrical signal Sig1 applied by the application unit 21. Then, the acquisition unit 22 calculates and acquires, based on the phase angle θ1 of the impedance Z1 thus obtained, a change rate D1 of the phase angle θ1 of the impedance Z1.

In this embodiment, the change rate D1 is the ratio of a measured value to a reference value with respect to a value (e.g., the phase angle θ1 in this example) concerning the impedance Z1 of the electrical signal Sig1. In other words, the change rate D1 is expressed as a value obtained by dividing a measured value of the phase angle θ1 of the impedance Z1 by a reference value. Also, in this embodiment, the reference value is a value (e.g., the phase angle θ1 in this example) concerning the impedance Z1 of the electrical signal Sig1 in a state where the organism A1 has not started to exercise yet.

In this case, the point in time when the organism A1 starts to exercise may be, for example, a point in time when a vibration sensor, attached along with the pair of electrodes 3 to the organism A1, detects a vibration equal to or greater than a threshold value. Alternatively, the point in time when the organism A1 starts to exercise may also be, for example, a point in time when a value (e.g., the phase angle θ1 in this example) concerning the impedance Z1 changes for the first time since the beginning of the measurement. Still alternatively, the point in time when the organism A1 starts to exercise may also be, for example, a point in time when a value (e.g., the phase angle θ1 in this example) concerning the impedance Z1 reaches a threshold value for the first time since the beginning of the measurement.

The change rates D1 of the phase angle θ1 of the impedance Z1 were obtained by the acquisition unit 22. Exemplary results thereof are shown in FIG. 4. Specifically, FIG. 4 shows the results obtained when a human being (as an exemplary organism A1) was made to do an exercise of ascending and descending the stairs for 12 minutes. In FIG. 4, the ordinate indicates the change rate D1 of the phase angle θ1 of the impedance Z1 and the abscissa indicates the measuring time. Also, in FIG. 4, the curve L1 shows the results obtained when the electrical signal Sig1 had a frequency of 100 Hz. The curve L2 shows the results obtained when the electrical signal Sig1 had a frequency of 1 kHz. The curve L3 shows the results obtained when the electrical signal Sig1 had a frequency of 10 kHz. The curve L4 shows the results obtained when the electrical signal Sig1 had a frequency of 100 kHz.

As shown in FIG. 4, the lower the frequency of the electrical signal Sig1 is, the more significantly the change rate D1 of the phase angle θ1 of the impedance Z1 tends to change with time. Among other things, the change is particularly significant when the frequency is 100 Hz. Also, when the electrical signal Sig1 has a frequency of 100 Hz, the point in time when the human being finishes the exercise generally agrees with a peak of the change rate D1 of the phase angle θ1 of the impedance Z1. In other words, when the electrical signal Sig1 has a frequency of 100 Hz, the human being’s (organism’s A1) exercise is more closely correlated to the change rate D1 of the phase angle θ1 of the impedance Z1.

The electrical signal Sig1 particularly preferably has a frequency of 100 Hz as described above but may also have a frequency equal to or higher than 50 Hz and equal to or lower than 200 Hz. In other words, a frequency at which the phase angle θ1 of the impedance Z1 and the change rate D1 of the phase angle θ1 may be measured with good accuracy falls within the range from 50 Hz to 200 Hz and is more preferably 100 Hz.

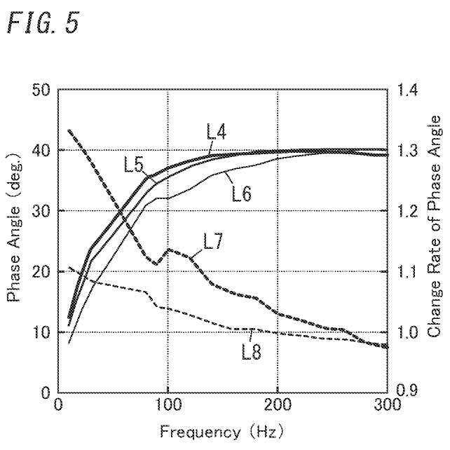

The reason will be described with reference to FIG. 5. FIG. 5 shows an exemplary correlation between the phase angle θ1 of the impedance Z1 and the frequency of the electrical signal Sig1 and an exemplary correlation between the change rate D1 of the phase angle θ1 of the impedance Z1 and the frequency of the electrical signal Sig1. In FIG. 5, the ordinate on the left indicates the phase angle θ1 of the impedance Z1, the ordinate on the right indicates the change rate D1 of the phase angle θ1 of the impedance Z1, and the abscissa indicates the frequency of the electrical signal Sig1. Also, in FIG. 5, the curves L4-L6 indicate the respective results of measurement of the phase angle θ1 of the impedance Z1. Specifically, the curve L4 indicates the result obtained immediately after the exercise finished, the curve L5 indicates the result obtained 30 minutes after the exercise finished, and the curve L6 indicates the result obtained 60 minutes after the exercise finished. Furthermore, in FIG. 5, the curves L7 and L8 indicate the results of measurement of the change rate D1 of the phase angle θ1 of the impedance Z1. Specifically, the curve L8 indicates the result obtained 30 minutes after the exercise finished and the curve L7 indicates the result obtained 60 minutes after the exercise finished.

As indicated by the curves L4-L6 in FIG. 5, the phase angle θ1 of the impedance Z1 decreases steeply once the frequency of the electrical signal Sig1 has become lower than 50 Hz. In other words, the phase angle θ1 of the impedance Z1 starts to increase when the frequency of the electrical signal Sig1 becomes equal to or higher than 50 Hz. Also, as indicated by the curves L7 and L8 in FIG. 5, once the frequency of the electrical signal Sig1 exceeds 200 Hz, the change rate D1 of the phase angle θ1 of the impedance Z1 changes much less significantly to become approximately equal to one. In other words, the change rate D1 of the phase angle θ1 of the impedance Z1 starts to increase when the frequency of the electrical signal Sig1 becomes equal to or lower than 200 Hz. Therefore, the electrical signal Sig1 preferably has a frequency equal to or higher than 50 Hz and equal to or lower than 200 Hz.

Then, the acquisition unit 22 acquires the biometric information based on the change rate D1 with respect to the impedance Z1 of the electrical signal Sig1 (e.g., the change rate D1 of the phase angle θ1 in this example). In this embodiment, the acquisition unit 22 calculates (i.e., the acquisition step ST2 includes calculating) the biometric information based on the change rate D1 with respect to the impedance Z1 of the electrical signal Sig1 (e.g., the change rate D1 of the phase angle θ1 in this example).

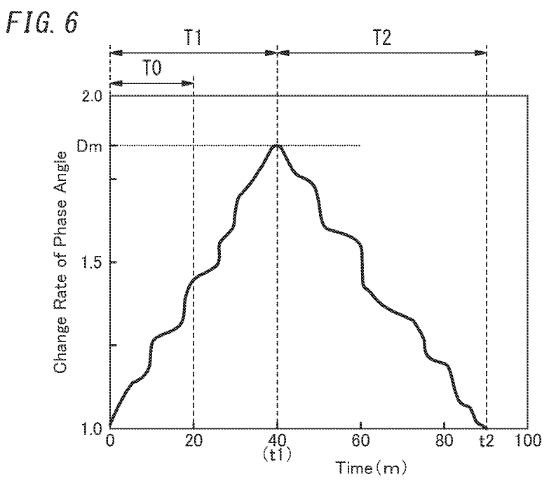

Next, a specific example of the biometric information which may be calculated based on the change rate D1 of the phase angle θ1 of the impedance Z1 will be described with reference to FIG. 6. The biometric information may include an activation rate P1 of the organism’s A1 muscle A11, a recovery rate P2 of the muscle A11, an amount of exercise J1 of the muscle A11, and an effect size E1 of the exercise done by the organism A1.

FIG. 6 conceptually shows an exemplary result obtained when a human being (organism A1) was made to do an exercise (such as jogging) for 20 minutes. In FIG. 6, the ordinate indicates the change rate D1 of the phase angle θ1 of the impedance Z1 and the abscissa indicates the measuring time. Also, in FIG. 6, T0 indicates an exercise period during which the human being is doing the exercise, T1 indicates an active period (to be described later), and T2 indicates a recovery period (to be described later). Furthermore, in FIG. 6, t1 indicates a point in time when the change rate D1 of the phase angle θ1 of the impedance Z1 reaches a maximum value Dm and t2 indicates a point in time when the change rate D1 of the phase angle θ1 of the impedance Z1 becomes equal to one.

The activation rate P1 of the muscle A11 is an index indicating to what degree the target muscle A11 has been activated by the exercise done by the human being (organism A1). The activation rate P1 is correlated to the exercise intensity (i.e., the magnitude of the load applied to the muscle A11 and the duration for which the load is applied to the muscle A11) and/or the muscle mass of the muscle A11. The activation rate P1 of the muscle A11 is given by the following Equation (1):

$$P 1 = \frac{d D 1}{\text{dt}}$$

The activation rate P1 of the muscle A11 may be calculated by, for example, obtaining the gradient of the change rate D1 of the phase angle θ1 of the impedance Z1 by the least squares method within the range of the active period T1. Alternatively, the activation rate P1 of the muscle A11 may also be calculated approximately by, for example, dividing, by the active period T1, a value obtained by subtracting one from the maximum value Dm of the change rate D1 of the phase angle θ1 of the impedance Z1.

The active period T1 as used herein refers to a period from a point in time when the human being (organism A1) starts doing an exercise to a point in time when the change rate D1 of the phase angle θ1 of the impedance Z1 reaches the maximum value Dm. That is to say, the active period T1 is not the period during which the human being is doing the exercise but corresponds to a period from the point in time when the human being started doing the exercise to a point in time when a certain time has passed since the human being finished doing the exercise.

The recovery rate P2 of the muscle A11 is an index indicating to what degree the target muscle A11 has recovered after the human being (organism A1) has finished doing the exercise. The recovery rate P2 is correlated to the degree to which the muscle A11 has been trained, the blood flow rate, and/or the human being’s physical condition. The recovery rate P2 of the muscle A11 is given by the following Equation (2):

$$P 2 = - \frac{d D 1}{d t}$$

The recovery rate P2 of the muscle A11 may be calculated by, for example, obtaining the gradient of the change rate D1 of the phase angle θ1 of the impedance Z1 by the least squares method within the range of the recovery period T2. Alternatively, the recovery rate P2 of the muscle A11 may also be calculated approximately by, for example, dividing, by the recovery period T2, a value obtained by subtracting one from the maximum value Dm of the change rate D1 of the phase angle θ1 of the impedance Z1.

The recovery period T2 as used herein refers to a period of time that it takes for the change rate D1 of the phase angle θ1 of the impedance Z1 to decrease from the maximum value Dm to one. That is to say, the recovery period T2 is not a period that begins from a point in time when the human being (organism A1) finishes doing the exercise but corresponds to a period that begins from a point in time when a certain time has passed since the human being finished doing the exercise.

The amount of exercise J1 of the muscle A11 is an index indicating to what degree the target muscle A11 has been activated by the exercise done by the human being (organism A1) and is correlated to the exercise intensity. The amount of exercise J1 of the muscle A11 is given by the following Equation (3):

$$J 1 = {\int }_{0}^{t 1} \left(D 1 - 1\right) d t$$

The effect size E1 of the exercise done by the organism A1 is an index indicating to what degree the condition of the muscle A11 has changed as a result of the exercise done by the human being (organism A1) and is correlated to the degree of swelling of the muscle A11. The effect size E1 of the muscle A11 is given by the following Equation (4):

$$E 1 = {\int }_{t 1}^{t 2} \left(D 1 - 1\right) d t$$

Alternatively, the amount of exercise J1 of the muscle A11 may also be calculated approximately by, for example, the following Equation (5). Also, the effect size E1 of the exercise done by the organism A1 may also be calculated approximately by, for example, the following Equation (6):

$$J 1 = \frac{1}{2} \left(D m - 1\right) ⋅ t 1$$

$$E 1 = \frac{1}{2} \left(D m - 1\right) ⋅ \left(t 2 - t 1\right)$$

As described above, according to this embodiment, not the period during which the human being (organism A1) is doing the exercise but the time it takes for the change rate D1 of the phase angle θ1 of the impedance Z1 to reach the maximum value Dm is used. Thus, this embodiment achieves the advantage of making it easier to accurately measure the amount of exercise of the target muscle A11 and/or the effect size of the exercise and eventually recognize the change in the condition of the muscle A11 quantitatively.

In addition, according to this embodiment, not the period during which the human being (organism A1) is doing the exercise but the gradient of the change rate D1 in a period during which the change rate D1 of the phase angle θ1 of the impedance Z1 increases from an initial value to the maximum value Dm and/or the gradient of the change rate D1 in a period during which the change rate D1 of the phase angle θ1 of the impedance Z1 decreases from the maximum value Dm to the initial value is/are used. Thus, this embodiment achieves the advantage of making it easier to accurately measure the activation rate P1 of the target muscle A11 and/or the recovery rate P2 of the muscle A11 and appropriately set the exercise intensity and/or the recovery period of the muscle A11.

The storage unit 4 includes an electrically programmable nonvolatile memory such as an electrically erasable programmable read-only memory (EEPROM) and a volatile memory such as a random-access memory (RAM). The storage unit 4 stores the biometric information acquired by the acquisition unit 22 with respect to each individual organism A1 as the target of measurement. In this embodiment, the storage unit 4 stores the results of calculation obtained by the acquisition unit 22 (namely, the activation rate P1 of the human being’s (organism’s A1) muscle A11, the recovery rate P2 of the muscle A11, the amount of exercise J1 of the muscle A11, and the effect size E1 of the exercise done by the human being) as biometric information with respect to each individual human being as the target of measurement.

The output unit 5 outputs either the biometric information acquired by the acquisition unit 22 or the biometric information stored in the storage unit 4 to an external device. The output unit 5 is an agent that performs the output step ST3 (see FIG. 7). That is to say, the output unit 5 outputs (i.e., the output step ST3 includes outputting) the results of calculation made by the acquisition unit 22 (in the acquisition step ST2). The output unit 5 may, for example, output the biometric information to a mobile communications device (such as a smartphone, a tablet computer, or a personal computer) by establishing wireless or wired communication with the mobile communications device. Alternatively, the output unit 5 may also have the biometric information displayed on, for example, a display device (such as a liquid crystal display or an organic electroluminescent (EL) display) provided for the measuring apparatus 10. Still alternatively, the output unit 5 may also have the biometric information emitted as a voice message from a loudspeaker provided for the measuring apparatus 10.

### Operation

Next, an exemplary operation of the biometric information acquisition system 100 according to this embodiment, i.e., an exemplary procedure of a series of processing steps of the biometric information acquisition method, will be described with reference to FIG. 7. In the following description, the pair of electrodes 3 are supposed to have already been attached to the human being’s (organism’s A1) muscle A11. First, the application unit 21 applies the electrical signal Sig1 to the muscle A11 via the pair of electrodes 3 (in S1). The processing step S1 corresponds to the application step ST1.

Next, the acquisition unit 22 performs the following processing steps S2-S4 corresponding to the acquisition step ST2. Specifically, the acquisition unit 22 measures a value (e.g., a phase angle θ1 in this example) concerning the impedance Z1 of the electrical signal Sig1 applied to the muscle A11 (in S2). Then, the acquisition unit 22 calculates and acquires, based on the phase angle θ1 of the impedance Z1 of the electrical signal Sig1, the change rate D1 of the phase angle θ1 of the impedance Z1 (in S3). Subsequently, the acquisition unit 22 calculates and acquires, based on the change rate D1 of the phase angle θ1 of the impedance Z1, biometric information such as the activation rate P1 of the muscle A11 (in S4).

Thereafter, the output unit 5 outputs the results of calculation made by the acquisition unit 22, i.e., outputs the biometric information (in S5). The processing step S5 corresponds to the output step ST3. The output unit 5 may output the biometric information either at regular intervals or in response to the request of the user of the biometric information acquisition system 100, whichever is appropriate. Note that the biometric information acquired by the acquisition unit 22 is stored in the storage unit 4 as well.

As can be seen from the foregoing description, this embodiment enables acquiring a parameter correlated to the muscle mass of the organism’s A1 muscle A11 by applying the electrical signal Sig1 to the organism’s A1 muscle A11 and acquiring the change rate D1 with respect to the impedance Z1 of the electrical signal Sig1. Thus, this embodiment achieves the advantage of making it easier to recognize a change in the organism’s A1 muscle A11. This embodiment may be used in any of the situations to be enumerated below. In each of the following situations, the organism A1 is a human being.

Firstly, in general, as a human being grows older, his or her muscle mass gradually decreases from his or her youth. If a person has no exercise habit, the decrease in the muscle mass may affect his or her daily life (e.g., may increase the chances of his or her stumbling, for example). Thus, it is preferable for a human being to develop a sufficient muscle mass before he or she reaches his or her middle age by establishing the exercise habit in his or her youth (i.e., to make so-called “muscle savings”). Also, to establish the exercise habit (i.e., to keep a person motivated to exercise on a regular basis), it is desirable to visualize the amount of exercise of the muscle.

This embodiment enables acquiring, as biometric information, the amount of exercise J1 and/or effect size E1 of the muscle A11 as described above. Thus, this embodiment may contribute to keeping a person motivated to do exercises on a regular basis by, for example, displaying, on a display device, the amount of exercise J1 and/or effect size E1 of the muscle A11 thus acquired.

Secondly, a person who has already established an exercise habit (such as a professional athlete) needs to apply a proper load to his or her muscles through daily training. That is to say, if a proper load is applied to the muscles, muscle swelling and/or increase in muscular strength would be expected. On the other hand, if the load applied to the muscles is insufficient, neither muscle swelling nor increase in muscular strength would be expected. Besides, an excessive load applied to the muscles (i.e., in cases of overtraining) could lead to muscle fatigue and/or decrease in muscular strength. This problem applies to not only professional athletes but also anybody who makes it a rule to do exercises on a daily basis. Therefore, it is desirable to visualize the amount of exercise of muscles and the status of recovery of muscles in order to apply a proper load to muscles.

This embodiment enables acquiring, as biometric information, the amount of exercise J1 of the muscle A11, the effect size E1 thereof, and the recovery rate P2 of the muscle A11 as described above. Thus, this embodiment may contribute to applying a proper load to the muscle A11 by, for example, displaying, on a display device, the amount of exercise J1 of the muscle A11, the effect size E1, and the recovery rate P2 of the muscle A11 that have been acquired.

Thirdly, it is known that if a person stays in bed for a long time (e.g., two weeks or more) due to illness, for example, then a cross-sectional area of his or her muscle fibers (including fast muscle fibers and slow muscle fibers) decreases by a few ten %. If the muscle fibers decrease to such a degree, he or she may fall into the condition of frailty. To prevent the person from falling into the condition of frailty, it is necessary for him or her to increase the muscle fibers (i.e., recover the muscles) after leaving his or her sickbed. Thus, it is desirable to present, in a visualized form, a proper amount of exercise and/or effect size of an exercise that needs to be done to recover the muscles but is easy to continue for a person without placing an excessive burden on him or her.

This embodiment enables acquiring, as biometric information, the amount of exercise J1 of the muscle A11 and/or effect size E1 thereof. Thus, this embodiment may contribute to presenting a proper amount of exercise that is easy to continue by, for example, displaying, on a display device, the amount of exercise J1 of the muscle A11 and/or the effect size E1 thereof that have been acquired.

### Variations

Note that the embodiment described above is only an exemplary one of various embodiments of the present disclosure and should not be construed as limiting. Rather, the exemplary embodiment may be readily modified in various manners depending on a design choice or any other factor without departing from the scope of the present disclosure. Also, the functions of the biometric information acquisition method may also be implemented as a (computer) program or a non-transitory storage medium on which the program is stored, for example. A (computer) program according to an aspect is designed to cause one or more processors to perform the biometric information acquisition method described above.

Next, variations of the exemplary embodiment will be enumerated one after another. Note that the variations to be described below may be adopted in combination as appropriate.

The biometric information acquisition system 100 according to the present disclosure includes a computer system. The computer system includes a processor and a memory as principal hardware components. The functions of the biometric information acquisition system 100 according to the present disclosure may be performed by making the processor execute a program stored in the memory of the computer system. The program may be stored in advance in the memory of the computer system. Alternatively, the program may also be downloaded through a telecommunications line or be distributed after having been recorded in some non-transitory storage medium such as a memory card, an optical disc, or a hard disk drive, any of which is readable for the computer system. The processor of the computer system may be made up of a single or a plurality of electronic circuits including a semiconductor integrated circuit (IC) or a large-scale integrated circuit (LSI). As used herein, the “integrated circuit” such as an IC or an LSI is called by a different name depending on the degree of integration thereof. Examples of the integrated circuits include a system LSI, a very-large-scale integrated circuit (VLSI), and an ultra-large-scale integrated circuit (ULSI). Optionally, a field-programmable gate array (FPGA) to be programmed after an LSI has been fabricated or a reconfigurable logic device allowing the connections or circuit sections inside of an LSI to be reconfigured may also be adopted as the processor. Those electronic circuits may be either integrated together on a single chip or distributed on multiple chips, whichever is appropriate. Those multiple chips may be aggregated together in a single device or distributed in multiple devices without limitation. As used herein, the “computer system” includes a microcontroller including one or more processors and one or more memories. Thus, the microcontroller may also be implemented as a single or a plurality of electronic circuits including a semiconductor integrated circuit or a large-scale integrated circuit.

Also, in the embodiment described above, the plurality of functions of the biometric information acquisition system 100 are aggregated together in a single housing. However, this is not an essential configuration for the biometric information acquisition system 100. Alternatively, those constituent elements of the biometric information acquisition system 100 may be distributed in multiple different housings. Still alternatively, at least some functions of the biometric information acquisition system 100 may be implemented as a cloud computing system as well.

In the embodiment described above, the organism’s A1 (e.g., the human being’s in this example) muscle A11 is supposed to be a gastrocnemius muscle. However, this is only an example and should not be construed as limiting. Alternatively, the organism’s A1 muscle A11 may also be, for example, a soleus muscle, a biceps femoris muscle, a psoas major muscle, or a gluteus maximus muscle. Furthermore, the muscle A11 does not have to be a muscle of the lower half of a human being’s body but may also be a muscle of the upper half of his or her body such as a biceps brachii muscle, a triceps brachii muscle, a latissimus dorsi muscle, or a trapezius muscle.

In the embodiment described above, the electrical signal Sig1 applied by the application unit 21 to the organism’s A1 muscle A11 has a single frequency. However, this is only an example and should not be construed as limiting. Alternatively, the frequency of the electrical signal Sig1 may also be varied during the measurement.

In the embodiment described above, the change rate D1 with respect to the impedance Z1 of the electrical signal Sig1 to be referred while the acquisition unit 22 is acquiring the biometric information is the change rate D1 of the phase angle θ1 of the impedance Z1. However, this is only an example and should not be construed as limiting. Alternatively, the change rate D1 with respect to the impedance Z1 of the electrical signal Sig1 may also be the change rate D1 of the impedance Z1 itself, for example.

In the embodiment described above, a reference value used to acquire the change rate D1 of the phase angle θ1 of the impedance Z1 is the phase angle θ1 of the impedance Z1 measured before the organism A1 starts to exercise. However, this is only an example and should not be construed as limiting. Alternatively, the reference value may also be a preset value.

In the embodiment described above, a pair of electrodes 3 are attached to the organism’s A1 muscle A11. However, the number of the electrodes 3 attached does not have to be two but may also be three or more, for example.

In the embodiment described above, the electrodes 3 attached to the organism’s A1 muscle A11 are made of a metal. Alternatively, the electrodes 3 may also be made of electrically conductive rubber, for example.

In the embodiment described above, the electrodes 3 attached to the organism’s A1 muscle A11 only need to make close contact with the skin surface. Thus, one surface, facing the skin surface, of the electrodes 3 does not have to have adhesiveness and does not have to be adhered to the skin surface, either.

### Recapitulation

As can be seen from the foregoing description, a biometric information acquisition method according to a first aspect includes an application step (ST1) and an acquisition step (ST2). The application step (ST1) includes applying an electrical signal (Sig1) to an organism’s (A1) muscle (A11). The acquisition step (ST2) includes acquiring information about the organism’s (A1) muscle (A11) as biometric information based on a change rate (D1) with respect to an impedance (Z1) of the electrical signal (Sig1) applied in the application step (ST1).

This aspect achieves the advantage of making it easier to recognize a change in the organism’s (A1) muscle (A11).

In a biometric information acquisition method according to a second aspect, which may be implemented in conjunction with the first aspect, the change rate (D1) is a ratio of a measured value to a reference value with respect to a value concerning the impedance (Z1) of the electrical signal (Sig1).

This aspect achieves the advantage of making it even easier to recognize a change in the organism’s (A1) muscle (A11).

In a biometric information acquisition method according to a third aspect, which may be implemented in conjunction with the second aspect, the reference value is a value concerning the impedance (Z1) of the electrical signal (Sig1) in a state where the organism (A1) has not started to exercise yet.

According to this aspect, the reference state is a state of the organism’s (A1) muscle (A11) before the organism (A1) starts to exercise, thus achieving the advantage of making it even easier to recognize a change in the organism’s (A1) muscle (A11) during or after the exercise.

In a biometric information acquisition method according to a fourth aspect, which may be implemented in conjunction with any one of the first to third aspects, the change rate (D1) is a rate of change (D1) in a phase angle (θ1) of the impedance (Z1) of the electrical signal (Sig1).

This aspect achieves the advantage of making it even easier to recognize a change in the organism’s (A1) muscle (A11) compared to a situation where the change is recognized based on the change rate (D1) of the impedance (Z1) itself of the electrical signal (Sig1).

In a biometric information acquisition method according to a fifth aspect, which may be implemented in conjunction with any one of the first to fourth aspects, the electrical signal (Sig1) has a frequency equal to or higher than 50 Hz and equal to or lower than 200 Hz.

This aspect achieves the advantage of making it easier to acquire the change rate (D1) with respect to the impedance (Z1) of the electrical signal (Sig1).

In a biometric information acquisition method according to a sixth aspect, which may be implemented in conjunction with any one of the first to fifth aspects, the application step (ST1) includes applying a pulse-modulated voltage as the electrical signal (Sig1) to the organism’s (A1) muscle (A11).

This aspect achieves the advantage of making it easier to obtain a peak value with respect to a value concerning the impedance (Z1) of the electrical signal (Sig1), compared to a situation where a sinusoidal AC voltage is applied.

In a biometric information acquisition method according to a seventh aspect, which may be implemented in conjunction with any one of the first to sixth aspects, the electrical signal (Sig1) has a single frequency.

This aspect achieves the advantage of making it easier to simplify a configuration for measuring a value concerning the impedance (Z1) of the electrical signal (Sig1), compared to a situation where the frequency of the electrical signal (Sig1) is allowed to vary.

In a biometric information acquisition method according to an eighth aspect, which may be implemented in conjunction with any one of the first to seventh aspects, the acquisition step (ST2) includes calculating the biometric information based on the change rate (D1) with respect to the impedance (Z1) of the electrical signal (Sig1). The biometric information acquisition method further includes an output step (ST3) including outputting a result of calculation obtained in the acquisition step (ST2).

This aspect achieves the advantage of making it easier to recognize a change in the organism’s (A1) muscle (A11).

In a biometric information acquisition method according to a ninth aspect, which may be implemented in conjunction with any one of the first to eighth aspects, the application step (ST1) includes applying the electrical signal (Sig1) to the organism’s (A1) muscle (A11) via a pair of electrodes (3).

This aspect achieves the advantage of making it easier to simplify a configuration for measuring a value concerning the impedance (Z1) of the electrical signal (Sig1), compared to a situation where three or more electrodes (3) are used.

A program according to a tenth aspect is designed to cause one or more processors to perform the biometric information acquisition method described above.

This aspect achieves the advantage of making it easier to recognize a change in the organism’s (A1) muscle (A11).

A biometric information acquisition system (100) according to an eleventh aspect includes an application unit (21) and an acquisition unit (22). The application unit (21) applies an electrical signal (Sig1) to an organism’s (A1) muscle (A11). The acquisition unit (22) acquires information about the organism’s (A1) muscle (A11) as biometric information based on a change rate (D1) with respect to an impedance (Z1) of the electrical signal (Sig1) applied by the application unit (21).

This aspect achieves the advantage of making it easier to recognize a change in the organism’s (A1) muscle (A11).

Note that the features of the methods according to the second to ninth aspects are not essential features for the biometric information acquisition method but may be omitted as appropriate.

Reference Signs List

| | |
|---|---|
| 100 | Biometric Information Acquisition System |
| 21 | Application Unit |
| 22 | Acquisition Unit |
| 3 | Electrode |
| A1 | Organism |
| A11 | Muscle |
| D1 | Change Rate |
| Sig1 | Electrical Signal |
| Z1 | Impedance |
| θ1 | Phase Angle |
| ST1 | Application Step |
| ST2 | Acquisition Step |
| ST3 | Output Step |

1. A biometric information acquisition method comprising:
an application step including applying an electrical signal to an organism’s muscle; and
an acquisition step including acquiring information about the organism’s muscle as biometric information based on a change rate with respect to an impedance of the electrical signal applied in the application step.

2. The biometric information acquisition method of claim 1, wherein
the change rate is a ratio of a measured value to a reference value with respect to a value concerning the impedance of the electrical signal.

3. The biometric information acquisition method of claim 2, wherein
the reference value is a value concerning the impedance of the electrical signal in a state where the organism has not started to exercise yet.

4. The biometric information acquisition method of claim 1, wherein
the change rate is a rate of change in a phase angle of the impedance of the electrical signal.

5. The biometric information acquisition method of claim 1, wherein
the electrical signal has a frequency equal to or higher than 50 Hz and equal to or lower than 200 Hz.

6. The biometric information acquisition method of claim 1, wherein
the application step includes applying a pulse-modulated voltage as the electrical signal to the organism’s muscle.

7. The biometric information acquisition method of claim 1, wherein
the electrical signal has a single frequency.

8. The biometric information acquisition method of claim 1, wherein
the acquisition step includes calculating the biometric information based on the change rate with respect to the impedance of the electrical signal, and
the biometric information acquisition method further includes an output step including outputting a result of calculation obtained in the acquisition step.

9. The biometric information acquisition method of claim 1, wherein
the application step includes applying the electrical signal to the organism’s muscle via a pair of electrodes.

10. A non-transitory storage medium storing a program that is designed to cause one or more processors to perform the biometric information acquisition method of claim 1.

11. A biometric information acquisition system comprising:
an application unit configured to apply an electrical signal to an organism’s muscle; and
an acquisition unit configured to acquire information about the organism’s muscle as biometric information based on a change rate with respect to an impedance of the electrical signal applied by the application unit.

12. The biometric information acquisition method of claim 2, wherein
the change rate is a rate of change in a phase angle of the impedance of the electrical signal.

13. The biometric information acquisition method of claim 3, wherein
the change rate is a rate of change in a phase angle of the impedance of the electrical signal.

14. The biometric information acquisition method of claim 2, wherein
the electrical signal has a frequency equal to or higher than 50 Hz and equal to or lower than 200 Hz.

15. The biometric information acquisition method of claim 3, wherein
the electrical signal has a frequency equal to or higher than 50 Hz and equal to or lower than 200 Hz.

16. The biometric information acquisition method of claim 4, wherein
the electrical signal has a frequency equal to or higher than 50 Hz and equal to or lower than 200 Hz.

17. The biometric information acquisition method of claim 2, wherein
the application step includes applying a pulse-modulated voltage as the electrical signal to the organism’s muscle.

18. The biometric information acquisition method of claim 3, wherein
the application step includes applying a pulse-modulated voltage as the electrical signal to the organism’s muscle.

19. The biometric information acquisition method of claim 4, wherein
the application step includes applying a pulse-modulated voltage as the electrical signal to the organism’s muscle.

20. The biometric information acquisition method of claim 5, wherein
the application step includes applying a pulse-modulated voltage as the electrical signal to the organism’s muscle.
